(19) United States
(12) Patent Application Publication
(10) Pub. No.: US 2022/0047727 A1
(43) Pub. Date: Feb. 17, 2022

(54) ULTRA-SMALL SUPERPARAMAGNETIC IRON OXIDE NANOPARTICLES

(71) Applicant: Saving Patients' Lives Medical B.V., Nijmegen (NL)

(72) Inventor: Robert de Boer, Nijmegen (NL)

(73) Assignee: Saving Patients' Lives Medical B.V., Nijmegen (NL)

(21) Appl. No.: 17/274,790

(22) PCT Filed: Sep. 10, 2019
(86) PCT No.: PCT/EP2019/074097

§ 371 Date: Mar. 10, 2021

(30) Foreign Application Priority Data
Sep. 10, 2018 (EP) 18193440.7

(51) Int. Cl. A61K 49/18; H01F 1/00; C01G 49/02

(52) U.S. Cl. CPC A61K 49/1863; B82Y 5/00; C01G 49/02; H01F 1/0018

(57) ABSTRACT

The invention relates to a method for producing an adjusted nanoparticle composition comprising ultra-small superparamagnetic iron oxide nanoparticles coated with dextran-T10, compositions obtained thereby, and uses of such compositions. The adjusted compositions have improved parameters such as lower batch-to-batch variance and improved stability, and are useful as magnetic imaging agents.

### FIELD OF THE INVENTION

The invention relates to a method for producing an adjusted nanoparticle composition comprising ultra-small superparamagnetic iron oxide nanoparticles coated with dextran-T10, compositions obtained thereby, and uses of such compositions. The adjusted compositions have improved parameters such as lower batch-to-batch variance and improved stability, and are useful as magnetic resonance imaging (MRI) agents.

### BACKGROUND ART

Iron oxide nanoparticles coated with dextran are useful as MRI contrast agents. An example is the drug product Ferumoxtran-10, which is also known by its brand names Sinerem and Combidex. It is a contrast agent from the category of the USPIO (ultra-small superparamagnetic iron oxide particles, sometimes also referred to as USPIONP) used in magnetic resonance imaging (MRI) as a marker of the reticuloendothelial system. It comprises nano-sized iron oxide particles, and it is intended for diagnostics of lymph node metastases in patients with malignant tumours. When used in combination with MRI, the particles help distinguish normal lymph nodes in the body from metastatic lymph nodes, i.e. lymph nodes which are infiltrated by malignant tumour metastases. Even very small lymph nodes (down to 2 mm diameter) can be detected using this technique. Drug products in this category are generally supplied as a lyophilizate which after reconstitution in water such as a 0.9% sodium chloride solution, or such as water for injection, result in a colloidal solution of iron oxide nanoparticles, or they are supplied as a liquid formulation. They are intended for parenteral use and administration as an infusion after dilution in 0.9% sodium chloride solution.

Dextran-coated iron oxide particles, their use, and their preparation are known in the art (EP0713602B1; U.S. Pat. No. 5,262,176; Corot et al., doi:10.1016/j.addr.2006.09.013; Saleh et al., doi:10.1002/nbm.881; Weinstein et al., doi:10.1038/jcbfm.2009.192; Laurent et al., doi: 10.1021/cr068445e). Ferumoxtran-10 has been clinically developed, for example in phase III clinical trials (Sigal et al., doi: 10.1007/s003300101130).

Jung and Jacobs (1995, Magnetic Resonance Imaging, Vol. 13, No. 5, pp. 661-674) report that the actual iron oxide core inside a Ferumoxtran-10 particle is composed of nonstoichiometric magnetite and has a mean diameter of 5.8-6.2 nm as measured by X-ray diffraction line broadening. The hydrodynamic diameter of a particle is about 21 nm (number weighted). It is discussed that a thicker dextran coating reduces the interaction of particles with plasma proteins and thus reduce opsonisation.

Jung (1995, Magnetic Resonance Imaging, Vol. 13, No. 5, pp. 675-691) further reports that no covalent interaction between dextran and the iron oxide particles occurs, and that thicker layers of dextran would further reduce opsonisation. The layer thickness for Ferumoxtran-10 is reported as 8-12 nm, corresponding to approximately 30 adsorbed dextran molecules per core. Dextran binding was found to be according to the classic Jenckel and Rumbach model of polymer adsorption on surfaces, involving reversible polymer-surface interactions with loops (non-adsorbed internal segments), trains (adsorbed internal segments), and tails (non-adsorbed end segments).

Anzai et al. (1994, AJNR Am J Neuroradiol 15:87-94) describe that Ferumoxtran-10 nanoparticles have longer plasma half-life (>200 minutes) than uncoated iron oxide USPIO despite the fact that the dextran coating increases particle size. This is because recognition of particles by macrophages does not only depend on particle size, but also on surface properties. Iron oxide itself can be either negatively or positively charged in solution, causing immobilization of water molecules around its surface. This results in gradual enlargement of the particle size during circulation. Dextran is an uncharged coating for the iron oxide particles and effectively stabilizes particle size in the vascular compartment. Thus, dextran-coated particles are not easily trapped by the mononuclear phagocytic system of liver and spleen. Particles such as Ferumoxtran-10 are biodegradable and are excreted from the body via the usual catabolic pathway.

Dextran is a complex branched glucan (a polysaccharide consisting of linked glucose molecules) composed of chains of varying lengths. It is often assigned a T-number, which generally corresponds to the average molecular weight in kDa. For example, dextran-T1 has an average molecular weight of about 1000 Da, and dextran-T10 has an average molecular weight of about 10000 Da or 10 kDa. In their application in Ferumoxtran-10, dextran must have a molecular weight sufficient to allow to control the size of the iron oxide cores in the step of precipitation of iron salts, and to aid to stabilise the particles during the process and in the drug product. The use of other polymers or macromolecules (starch, chondroitin, or glucosaminoglycane from WO 97/25073, Heparin from WO 96/09840) is described in the literature, but no major advantages are reported.

Unterweger et al. (doi: 10.2147/IJN.S138108) report that iron oxide nanoparticles coated with dextran-T40 (having an average molecular weight of about 40 kDa) are characterized by a narrow size distribution of about 80 nm. The use of dextran-T40 during particle production leads to particles having less immunogenic properties.

Paul et al. (doi: 10.1021/bc034194u) report that iron oxide nanoparticles coated with dextran-T10 have a dextran-to-iron content of about 0.79 gram bound dextran per gram of iron. It is further reported that the use of dextran with a lower chain length than dextran-T10 (namely dextran-T1 and dextran-T5) leads to iron oxide particles with inferior magnetic properties, as colloidal material could not be obtained. The report recommends the use of reduced dextrans during production to improve properties of the resulting particles.

Bourrinet (presentation “Congrès de la SFT”, 20-21 Oct. 2008, Paris) disclosed a formulation of iron oxide nanoparticles having a dextran-T10 coating, comprising the additional excipients of 150 wt.-% dextran-T10, 150 wt.-% dextran-T1, and 9.5 wt.-% citrate, each relative to the iron content.

There is an ongoing need for more reliable production methods for dextran-coated ultra-small superparamagnetic iron oxide nanoparticles (dUSPIO). Accordingly, there is an ongoing need for compositions of such particles with more reliable characteristics.

### SUMMARY OF THE INVENTION

In a first aspect, the invention provides a method for producing an adjusted nanoparticle composition, the method comprising the steps of:

i) providing an ultrafiltrated composition comprising ultra-small superparamagnetic iron oxide nanoparticles coated with dextran-T10;

ii) determining the amount of dextran-T10 present in the ultrafiltrated composition of step i);

iii) adding an amount of dextran-T10 to the ultrafiltrated composition of step i) to obtain an adjusted composition comprising 140-160 wt.-% of dextran-T10 relative to the wt.-% of iron; and

iv) optionally filtering the adjusted composition.

In preferred embodiments, the ultrafiltrated composition of step i) or the adjusted composition of step iii) comprises from 1.5-2.5 wt.-% iron. In preferred embodiments, step ii) further comprises determining the amount of iron in the ultrafiltrated composition of step i). In preferred embodiments, step iii) further comprises adding a tonicity agent such as a citrate to the ultrafiltrated composition or to the adjusted composition, preferably in an amount of 7-12 wt.-% relative to the wt.-% of iron. In preferred embodiments, the ultrafiltrated composition of step i) further comprises a pharmaceutically acceptable excipient. In preferred embodiments, a filtrate of the ultrafiltrated composition of step i) has a conductivity of less than 500 μS/cm, preferably of less than 50 μS/cm, more preferably of less than 25 μS/cm.

In preferred embodiments, step iii) further comprises adding an amount of dextran-T1 to the ultrafiltrated composition or to the adjusted composition, so that the adjusted composition comprises 140-160 wt.-% dextran-T1 relative to the wt.-% of iron. In preferred embodiments, the adjusted composition comprises a substantially equal amount by weight of dextran-T10 and of dextran-T1. In preferred embodiments, the method further comprises the step of lyophilizing the adjusted composition.

In preferred embodiments, in step i) the ultrafiltrated composition is provided by a method comprising the steps of:

i.a) providing a solution of dextran-T10, FeCl₃, and FeCl₂ in water;

i.b) adding a base such as ammonium hydroxide to the solution of step i.a);

i.c) heating the solution of step i.b) to over 60° C. to obtain a nanoparticle composition comprising ultra-small superparamagnetic iron oxide nanoparticles coated with dextran-T10;

i.d) purifying the nanoparticle composition of step i.c) using ultrafiltration to obtain an ultrafiltrated composition.

In a second aspect, the invention provides a composition comprising:

i) ultra-small superparamagnetic iron oxide nanoparticles coated with dextran-T10;

ii) free dextran-T10;

iii) optionally a tonicity agent such as a citrate;
wherein the composition comprises 140-160 wt.-% of dextran-T10 relative to the wt.-% of iron.

In preferred embodiments, this composition is obtainable via a method according to the first aspect of the invention. In preferred embodiments, the concentration of dextran-T10 has a dispersion of at most ±10%. In preferred embodiments, the unimodal mean diameter of the nanoparticles is stable for at least 6 months. In a third aspect, the invention provides the composition for use as a medicament, wherein the medicament if preferably for in vivo diagnostics, more preferably for use as an MRI contrast agent.

### DESCRIPTION OF EMBODIMENTS

The inventors have surprisingly found a new production method for dextran-coated ultra-small superparamagnetic iron oxide nanoparticles (dUSPIO), the method including an important adjusting step. The resulting adjusted nanoparticles have improved characteristics.

### Method

Accordingly, the invention provides a method for producing an adjusted nanoparticle composition, the method comprising the steps of:

i) providing an ultrafiltrated composition comprising ultra-small superparamagnetic iron oxide nanoparticles coated with dextran-T10;

ii) determining the amount of dextran-T10 present in the ultrafiltrated composition of step i);

iii) adding an amount of dextran-T10 to the ultrafiltrated composition of step i) to obtain an adjusted composition comprising 140-160 wt.-% of dextran-T10 relative to the wt.-% of iron; and

iv) optionally filtering the adjusted composition.

Such a method is referred to hereinafter as a method according to the invention.

i) Provision of Unadjusted Nanoparticles

In step i) an ultrafiltrated composition is provided which comprises ultra-small superparamagnetic iron oxide nanoparticles coated with dextran-T10. Ultra-small superparamagnetic iron oxide nanoparticles are known in the art, see for example Merup & Hansen (2007) “Superparamagnetic particles”, doi: 10.1002/9780470022184.hmm409, in Handbook of Magnetism and Advanced Magnetic Materials. The ultrafiltrated composition is preferably an unadjusted composition, which is to say that it is preferably not a composition that has already been subjected to steps ii, iii, and iv as described above. As used herein, the term “unadjusted” refers to a composition or to nanoparticles that are not the product of a method according to the invention. These are known in the art, and the provision can be in any form, for example by de novo production of the particles, or by purchase from a commercial supplier, or by ultrafiltration of a nanoparticle composition to obtain an ultrafiltrated composition. Very suitable ultrafiltrated compositions are Sinerem or Ferumoxtran-10 or Combidex as described above.

dUSPIO are ultra-small nanoparticles, which are nanoparticles having a core diameter of less than about 20 nm. Preferred ultra-small nanoparticles have a core diameter of about 1 nm to about 20 nm, more preferably of about 1 nm to about 10 nm, more preferably of about 1 nm to about 8 nm, even more preferably of about 2 nm to about 8 nm, most preferably of about 4-6 nm or of about 5-7 nm or of about 4.3-5.6 nm. Preferred core diameters are no smaller than 1, 1.5, 2, 2.5, 3, 3.5, 4, 4.5, 5, 5.5, 6, 6.5, 7, 7.5, 8, 8.5, 9, 9.5, or 10 nm, preferably than 3, 3.25, 3.5, 3.75, 4, 4.25, or 4.5 nm. Preferred core diameters are no larger than 1.5, 2, 2.5, 3, 3.5, 4, 4.5, 5, 5.5, 6, 6.5, 7, 7.5, 8, 8.5, 9, 9.5, 10, or 10.5 nm, preferably than 5, 5.25, 5.5, 5.75, or 6 nm. As used herein, a core diameter is the diameter of the iron oxide comprised in the dUSPIO, preferably as determined by dynamic light scattering (DLS), transmission electron microscopy, or X-ray diffraction, such as for example described in Jung (1995) or Jung and Jacobs (1995). Preferably, a diameter as used in this document is a mean diameter. A preferred core diameter is a volume-weighted mean diameter. Preferably, a dUSPIO for use in a method according to the invention has substantially similar length, width, and height, preferably none of the three dimensions is more than 100, 80, 60, 50, 40, 30, 20, or 10% longer or shorter than one or two of the other three dimensions.

dUSPIO comprise a superparamagnetic iron oxide core. This core is preferably an iron oxide particle such as a crystal, for example a nanocrystal of iron oxide. A preferred crystal is a crystal that has a reverse spinel structure. Suitable iron oxides are oxides of Fe(II) such as FeO and FeO₂, oxides of Fe(III) such as α-Fe₂O₃ (hematite), β-Fe₂O₃, γ-Fe₂O₃ (maghemite), and ε-Fe₂O₃, and mixed oxides of Fe(II) and Fe(III) such as Fe₃O₄ (magnetite), Fe₄O₅, Fe₅O₆, Fe₅O₇, Fe₂₅O₃₂, and Fe₁₃O₁₉. Preferred iron oxides are oxides of Fe(III) and mixed oxides of Fe(II) and Fe(III). More preferred iron oxides are Fe₂O₃, Fe₃O₄, Fe₄O₅, Fe₅O₆, and Fe₅O₇. Even more preferred iron oxides are maghemite and magnetite, and magnetite is the most preferred iron oxide. A preferred magnetite is nonstoichiometric magnetite.

dUSPIO are preferably coated with dextran-T10. Naturally occurring dextran is a complex branched polysaccharide comprising a plurality of glucose molecules in chains of varying lengths, having a molecular weight of from about 3 to about 2000 kilodaltons (kDa). Dextran can also be of synthetic origin, in which case branching can be less prominent or even substantially absent or fully absent. As is known in the art, the average molecular weight (Mw) of dextran is often denoted as a T-number appended to the name, wherein the number refers to the average molecular weight in kDa. For example, dextran-T1 has an Mw of 1 kDa (or preferably of 800 to 1200 Da, more preferably of 850 to 1150 Da), dextran-T5 has an Mw of 5 kDa (or of 4500 to 5500 Da), and dextran-T10 has an Mw of 10 kDa (or of 9000 to 11000 Da). Preferred dextran-T10 for use in the invention has a specific rotation (+/−)° of +188 to +198; preferred dextran-T10 for use in the invention has an Mw of 9000 to 11000 Da; preferred dextran-T10 for use in the invention comprises at most 110 ppm nitrogen containing substances; preferred dextran-T10 for use in the invention has a loss on drying (105° C., 5 hours, in weight-% (wt.-%)) of at most 7; preferred dextran-T10 for use in the invention comprises less than 0.3 wt.-% sulphated ash; preferred dextran-T10 for use in the invention comprises fewer than 100 colony forming units per gram of microbial contamination; preferred dextran-T10 for use in the invention is of pharmaceutical quality (more preferably compliant with European Pharmacopoeia specifications except for molecular weight). Methods for determining these parameters are known in the art.

As used herein, the term “coated” refers to a layer of material that is associated with the outer surface of the iron oxide core. A coating is preferably substantially homogeneously distributed over the core surface. Coating thickness for dUSPIO is preferably determined by dynamic light scattering in aqueous solution, more preferably as described by Jung (1995) or Jung and Jacobs (1995). A preferred coating thickness is about 4-30 nm, more preferably about 5-25 nm, even more preferably about 6-20 nm, most preferably about 8-12 nm, corresponding to approximately 30 adsorbed dextran molecules per core. Preferred coatings have a thickness of at least about 4, 5, 6, 7, 8, 9, 10, 11, 12, 13, 14, 15, 16, 17, 18, 19, 20, 21, 22, 23, 24, 25, 26, 27, 28, or 29 nm, preferably of at least about 4, 5, 6, 7, 8, 9, or 10 nm, such as 7, 8, 9, or 10 nm. Preferred coatings have a thickness of at most about 5, 6, 7, 8, 9, 10, 11, 12, 13, 14, 15, 16, 17, 18, 19, 20, 21, 22, 23, 24, 25, 26, 27, 28, 29, or 30 nm, preferably of about 10, 11, 12, 13, 14, 15, 16, 17, 18, 19, 20, 21, 22, 23, 24, 25 nm, such as 10, 11, 12, 13, 14, 15, 16, or 17 nm. Preferably, cores have an average of about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 adsorbed dextran molecules. Preferably, dextran binding in a coating is according to the classic Jenckel and Rumbach model of polymer adsorption on surfaces.

The total diameter of dUSPIO is the sum of the core diameter and double the coating thickness. Preferred dUSPIO have a total diameter of about 9 to about 80 nm, more preferably of about 15 to about 50 nm, even more preferably of about 17 to about 25 nm, most preferably of about 18 to about 22 nm, such as 19 or 20 nm. Preferred dUSPIO have a total diameter of at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nm, preferably of at least 15, 16, 17, 18, 19, 20, 21, 22, 23, 24, 25, 26, 27, 28, 29, 30, 31, 32, 33, 34, or 35 nm, more preferably of at least 19, 20, 21, 22, 23, 24, 25, 26, 27, 28, 29, or 30 nm, such as 19 nm or 20 nm or 21 nm. Preferred dUSPIO have a total diameter of at most 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nm, preferably of at least 21, 22, 23, 24, 25, 26, 27, 28, 29, 30, 31, 32, 33, 34, 35, 36, 37, 38, 39, 40, 41, 42, 43, 44, or 45 nm, more preferably of at least 21, 22, 23, 24, 25, 26, 27, 28, 29, 30, or 31 nm, such as 29 nm or 30 nm or 31 nm.

As used herein, an ultrafiltrated composition is a composition that has been subjected to ultrafiltration. Ultrafiltration is routinely used in the art for purifying and/or concentrating macromolecules or nanoparticles. It is a type of membrane filtration wherein forces such as pressure lead to a separation through a semipermeable membrane. Suspended solids and solutes of high molecular weight, generally substances with a molecular weight that is higher than the filter molecular weight cutoff (MWCO), are retained in the retentate. Other substances such as solvent and low molecular weight solutes pass through the membrane and form the filtrate, also referred to as the permeate. Generally, these are substances with a molecular weight that is lower than the filter MWCO. Ultrafiltration is preferably tangential flow filtration (also known as crossflow filtration) or dead-end filtration, more preferably it is tangential flow filtration.

As used herein, ultrafiltration preferably uses a filter with an MWCO of about 30-400 kDa, more preferably of about 50-250 kDa, even more preferably of about 50-200 kDa, most preferably of about 80-150 kDa such as 100 kDa. An MWCO of a filter is preferably at least 30, 40, 50, 60, 70, 80, 90, 100, 110, 120, 130, 140, or 150 kDa, more preferably at least 50, 60, 70 80, 90, 100, 110, or 120 kDa, most preferably at least 90, 100, or 110 kDa. An MWCO of a filter is preferably at most 100, 110, 120, 130, 140, 150, 200, 250, 300, 350, or 400 kDa, more preferably at most 100, 110, 120, 130, 140, 150, 200, or 250 kDa, most preferably at most 100, 110, or 120 kDa.

In preferred embodiments, the unadjusted composition is produced as part of the method according to the invention. Accordingly, in preferred embodiments the invention provides the method according to the invention, wherein in step i) the ultrafiltrated composition is provided by a method comprising the steps of:

i.a) providing a solution of dextran-T10, FeCl₃, and FeCl₂ in water;

i.b) adding a base such as ammonium hydroxide to the solution of step i.a);

i.c) heating the solution of step i.b) to over 60° C. to obtain a nanoparticle composition comprising ultra-small superparamagnetic iron oxide nanoparticles coated with dextran-T10;

i.d) purifying the nanoparticle composition of step i.c) using ultrafiltration to obtain an ultrafiltrated composition.

In step i.a) constitutive materials for the eventual nanoparticles are provided. Step i.a) is preferably preformed in an inert atmosphere, such as under nitrogen, under helium, or under argon.

The solution can be stored at about 1-10° C., preferably at about 1-8° C. Water as used herein is preferably ultrapure water or water for injection. Preferably, the solvents of the solution of step i.a) comprise at least 70 wt.-% of water, more preferably at least 80, 90, 95, 96, 97, 98, 99, wt.-% of water. In highly preferred embodiments, water is the only solvent for the solution of step i.a).

The solution of dextran-T10, FeCl₃, and FeCl₂ in water is preferably obtained by mixing distinct solutions comprising at least one of dextran-T10, FeCl₃, and FeCl₂. Preferably, an FeCl₃ solution is added to a dextran-T10 solution, after which an aqueous FeCl₂ solution is additionally added. Preferably, each solution is filtered prior to use, such as through a 0.45 μm filter, a 0.2 μm filter, or both. Preferably, when two or more solutions are mixed, the resulting solution is stirred for at least 60, 70, 80, 90, 100, 110, 120, 130, 140, 150, 160, 170, 180, 190, 200, or 210 minutes, preferably for at least 110 minutes. This stirring is preferably performed at least within step i.a).

In this context, a preferred dextran-T10 solution comprises, preferably consists of water and dextran-T10, preferably comprising about 50-600 gram/liter of dextran-T10; more preferably, the solution comprises about 200-600 gram/liter of dextran-T10; even more preferably, the solution comprises about 400-600 gram/liter of dextran-T10; still more preferably, the solution comprises about 500-600 gram/liter of dextran-T10; most preferably the solution comprises about 550-580 gram/liter of dextran-T10. Preferably the solution is prepared by adding dextran-T10 to water, preferably under stirring. After preparation, the solution is optionally filtered, such as through a 0.45 μm filter, a 0.2 μm filter, or both. Laurent et al. suggest that use of dextran-T10 helps direct the size of the ultra-small superparamagnetic iron oxide nanoparticles that will be formed. To reach the desired 140-160 wt.-% dextran-T10 (relative to the wt.-% of iron) for compositions of the present invention, removal of excess dextran-T10 is required at a later stage.

In this context, a preferred FeCl₃ solution comprises or consists of water and FeCl₃, optionally also comprising dextran-T10, preferably as described above for a dextran-T10 solution. The solution preferably comprises about 4-120 gram/liter of FeCl₃; more preferably the solution comprises about 20-80 gram/liter of FeCl₃; even more preferably the solution comprises about 30-60 gram/liter of FeCl₃; most preferably the solution comprises about 40-45 gram/liter of FeCl₃, such as about 42 gram/liter of FeCl₃. A FeCl₃ solution is preferably prepared by addition of FeCl₃ such as its powder to a dextran-T10 solution as described above, preferably followed by at least 60, 90, 120, 150, or 180 minutes of stirring, such as by 180 minutes of stirring. In alternate preferred embodiments, the FeCl₃ is added as an aqueous solution of FeCl₃. Any addition of FeCl₃ to a solution, or any addition of a FeCl₃ solution to another solution, or any storage of FeCl₃ is preferably performed under an inert atmosphere such as an N₂ atmosphere, and preferably at about 1-6° C. A preferred FeCl₃ is anhydrous FeCl_{3.} A preferred FeCl₃ is FeCl₃ hexahydrate, more preferably FeCl₃ hexahydrate Ph. Eur. After preparation, the solution is optionally filtered, such as through a 0.45 μm filter, a 0.2 μm filter, or both.

In this context, a preferred FeCl₂ solution comprises or consists of water and FeCl₂, optionally also comprising dextran-T10, preferably as described above for a dextran-T10 solution. The solution preferably comprises about 10-1200 gram/liter of FeCl₂; more preferably the solution comprises about 70-1200 gram/liter of FeCl₂; even more preferably the solution comprises about 400-1000 gram/liter of FeCl₂; most preferably the solution comprises about 700-800 gram/liter of FeCl₂, such as about 740 gram/liter of FeCl₂. A FeCl₂ solution is preferably prepared by addition of FeCl₂ such as its powder to water, preferably followed by at least 30, 40, 50, 60, 70, 80, 90, 100, 100, or 120 minutes of stirring, such as by 110 minutes of stirring. Any addition of FeCl₂ to a solution, or any addition of a FeCl₂ solution to another solution, or any storage of FeCl₂ is preferably performed under an inert atmosphere such as an N₂ atmosphere, and preferably at about 2-8° C. A preferred FeCl₂ is anhydrous FeCl₂, FeCl₂ dihydrate, FeCl₂ tetrahydrate, or FeCl₂ hexahydrate. A more preferred FeCl₂ is FeCl₂ tetrahydrate. After preparation, the solution is optionally filtered, such as through a 0.45 μm filter, a 0.2 μm filter, or both.

In this context, a preferred solution of dextran-T10, FeCl₃, and FeCl₂ in water is provided by first preparing a dextran-T10 solution as described above, preferably by using about 800-1400 gram of dextran-T10, more preferably by using about 1100-1200 gram of dextran-T10, such as about 1143 gram, after which FeCl₃, preferably FeCl₃ hexahydrate, is added, preferably about 40-120 gram, more preferably about 70-100 gram, such as about 85 gram, after which the solution is mixed, preferably for at least 180 minutes, and then cooled under inert atmosphere such as N₂ atmosphere until it is 1 to 6° C. To this solution, a FeCl₂ solution is added, preferably under stirring and under an inert atmosphere such as an N₂ atmosphere. This FeCl₂ solution is preferably prepared as described above by using about 20 to 50 gram, preferably about 30 to 40 gram such as about 35 gram of FeCl₂ tetrahydrate and by stirring it, preferably for at least 110 minutes, after which the solution is cooled under inert atmosphere such as N₂ atmosphere until it is 2 to 8° C. The two separate solutions are then preferably filtered such as through a 0.45 μm filter, a 0.2 μm filter, or both, after which the FeCl₂ solution is added to the solution comprising dextran-T10 and FeCl₃, thus providing a solution of dextran-T10, FeCl₃, and FeCl₂ in water.

A solution of dextran-T10, FeCl₃, and FeCl₂ in water provided in step i) preferably has a ratio by weight of dextran-T10: FeCl₃: FeCl₂ in the range of 20-50:1-5:1, more preferably it is in the range of 20-40:2-4:1, even more preferably it is in the range of 30-35:2-3:1. Most preferably it is about 32.8:2.4:1. An example of such a ratio by weight is 1143:84.7:34.8.

It is to be understood that compound quantities mentioned throughout this description for use in method steps, and not mentioned as characteristics of a resulting product, are exemplary and are not intended to be limiting. For example amounts can be increased or decreased, preferably across a full process while maintaining described ratios.

In step i.b) a base is added to the solution of step i.a). Preferably, the base is added as a solution of that base, more preferably as an aqueous solution of that base. Suitable bases are carbonates, bicarbonates, hydroxides, and ammonia, such as NaOH, KOH, Na₂CO₃, NaHCO₃, CaCO₃, NH₃, and NH₄OH. Bases are preferably used as a solution of about 10 to 40 wt.-% in water, more preferably of about 20 to 35 wt.-% in water, even more preferably of about 25 to 30 wt.-% in water. Ammonia is a preferred base. Accordingly, for step i.b) it is highly preferred that an ammonia solution, preferably an NH₄OH solution of about 25 to 30 wt.-% in water is added to the solution of step i.a). It is even more preferred when such an ammonia solution is of pharmaceutical grade, and optionally is what is referred to in the art as a strong ammonia solution. When stored, such a solution is preferably stored at 2 to 8° C. When amounts in step i.a) are as described above, it is preferred to use about 10 to 1000 gram of base solution, more preferably about 50 to 500 gram, even more preferably about 75 to 150 gram, most preferably about 90 to 115 gram, such as about 103 gram. In other cases, the same ratio as described here is preferred.

In step i.c) the solution comprising dextran-T10, FeCl₃, FeCl₂, and base is heated to over 60° C. Preferably, it is heated to over 62, 64, 66, 68, 70, 72, 74, 76, 78, 80, 82, 84, 86, 88, 90, 92, 94, 96, 98, or 100° C. Preferably, it is not heated to more than 80, 82, 84, 86, 88, 90, 92, 94, 96, 98, 100, 102, 104, 106, 108, 110, 112, 114, 116, 118, or 120° C. Preferably, the solution is heated to about 60 to 100° C., more preferably to about 70 to 90° C., most preferably to about 75 to 85° C., such as to about 80° C. The heating is preferably performed under stirring, and preferably under an inert atmosphere such as an N₂ atmosphere. The heating as described above leads to formation of a nanoparticle composition comprising ultra-small superparamagnetic iron oxide nanoparticles coated with dextran-T10 (dUSPIO).

In step i.d) the nanoparticle composition of step i.c) is purified using ultrafiltration to obtain an ultrafiltrated composition. In this step, optionally the nanoparticle composition of step i.c) is also concentrated, or optionally is concentrated instead of purified. Preferably, prior to ultrafiltration the nanoparticle composition is diluted, preferably using a diluent which is a solvent that is compatible with ultrafiltration, such as water. The water for dilution is preferably at about the same temperature as the nanoparticle composition while it is added to the nanoparticle composition. The dilution is preferably about 2, 3, 4, 5, 6, 7, 8, 9, or 10-fold, more preferably about 3, 4, 5, 6, or 7-fold, most preferably about 4, 5, or 6-fold, such as 5-fold. Accordingly, preferably about 2, 3, 4, 5, 6, 7, 8, 9, or 10 volumes of diluent are added, more preferably about 2, 3, 4, 5, 6, or 7 volumes of diluent are added, even more preferably about 3, 4, or 5 volumes of diluent are added such as about 4 volumes of diluent. Prior to ultrafiltration the nanoparticle composition is preferably cooled, such as to about 1 to 60° C., preferably about 10 to 50° C., more preferably to about 15 to 35° C., most preferably to about 20 to 30° C. such as to about 25° C. Prior to ultrafiltration, preferably after cooling, the nanoparticle composition is preferably filtered such as through a 0.45 μm filter, a 0.2 μm filter, or both. Optionally, prior to ultrafiltration, multiple batches of nanoparticle compositions are combined, such as 2, 3, 4, 5, 6, 7, 8, 9, 10, 11, or 12 batches, preferably such as 3, 4, 5, 6, 7, 8, or 9 batches, more preferably 5, 6, or 7 batches such as 6 batches are combined.

The ultrafiltration preferably uses a filter with an MWCO of about 30-400 kDa, more preferably of about 50-250 kDa, even more preferably of about 50-200 kDa, most preferably of about 80-150 kDa such as 100 kDa. Preferably it is constant flow ultrafiltration such as tangential flow ultrafiltration. Preferably, filtration is continued until the volume of the composition has been reduced to about 20-80%, preferably to about 30-70%, more preferably to about 35-45% such as to about 40%. This is referred to herein as an ultrafiltration step. The filtrate is then optionally discarded after each ultrafiltration step.

As known to a skilled person, ultrafiltration can be performed batch-wise and continuously. Batch-wise ultrafiltration comprises adding certain volumes to the product to be ultrafiltrated. Continuous ultrafiltration comprises adding additional volumes (for example of buffers or other solutions) to the product whilst it is being ultrafiltrated during a certain period of time. In continuous ultrafiltration the volume of the composition is preferably kept stable, or substantially stable. A combination of both can be applied, resulting in the same product.

Optionally, after an ultrafiltration step, an additional ultrafiltration step is performed. Preferably, prior to such an additional ultrafiltration step, the nanoparticle composition is diluted with 1, 2, 3, 4, or 5 volumes of diluent, more preferably with 1, 2, or 3 volumes of diluent such as with 2 volumes of diluent. If such an additional ultrafiltration step is performed it is preferred when filtration is continued until the volume of the composition has been reduced to about 5-60%, preferably to about 8-30%, more preferably to about 10-15% such as to about 12%.

After such an additional ultrafiltration step, it is preferred that optional further additional ultrafiltration steps are performed. Prior to such further additional ultrafiltration steps, the nanoparticle composition is preferably diluted with about 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, or 5 volumes of diluent, more preferably with about 1.4, 1.6, 1.8, 2, 2.2, 2.4, 2.6, 2.8, 3, 3.2, or 3.4 volumes of diluent such as with about 2.6 volumes of diluent. Preferably, 1, 2, 3, 4, 5, 6, or more of such further additional ultrafiltration steps are performed, such as 3 further additional ultrafiltration steps. It is very preferable when the final ultrafiltration step that is performed reduces the volume of the ultrafiltrated nanoparticle composition to about 3-30%, preferably to about 5-15%, most preferably to about 5-10% such as to about 6.5%

Preferably, in total at least 2, 3, 4, 5, 6, or more ultrafiltration steps are performed. Preferably, additional ultrafiltration steps are performed until either a minimal total of diluent or washing fluid volumes such as 6, 7, 8, 9, 10, 11, 12, 13, 14, 15, or more, preferably 10 volumes have been used, or until the conductivity of the filtrate is sufficiently low, such as less than 500 μS/cm, preferably less than 100 μS/cm, more preferably less than 50 μS/cm, even more preferably less than 40 μS/cm, most preferably less than 25 μS/cm. Preferably, additional ultrafiltration steps are performed until both the minimal total of diluent or washing fluent volumes have been used and the conductivity of the filtrate is sufficiently low. Diluents are suitable washing fluids. Conductivity can be measured using an electrical conductivity meter, preferably at room temperature. In preferred embodiments is provided the method according to the invention, wherein a filtrate of the ultrafiltrated composition of step i) has a conductivity of less than 500 μS/cm, preferably of less than 100 μS/cm, more preferably of less than 50 μS/cm, even more preferably of less than 40 μS/cm, most preferably of less than 25 μS/cm.

Alternately, a single ultrafiltration step is performed wherein at least about 6, 7, 8, 9, 10, 11, 12, 13, 14, 15, or more volumes of washing fluid are used, preferably at least about 10 volumes, preferably at most about 20 volumes, more preferably at most about 15 volumes.

After ultrafiltration, the nanoparticle composition can be diluted using 1, 2, 3, 4, 5, 6, 7, 8, 9, or 10 volumes of diluent such as water. After removal of the nanoparticle composition from the ultrafiltration device, it is useful to rinse the ultrafiltration device using diluent, which can then be added to the nanoparticle composition; this improves yield of the nanoparticles. Preferably, the nanoparticle composition for use in step ii) is diluted using 4 additional volumes of diluent. Preferably, the ultrafiltration device is rinsed using about 1, 1.2, 1.4, 1.6, 1.8, 2, 2.2, 2.4, 2.6, 2.8, or 3 volumes of diluent, such as about 1.2 to 1.6 volumes, more preferably about 1.4 volumes; after this rinsing that volume is preferably added to the ultrafiltrated nanoparticle composition. After addition of such rinsing volumes it is preferred to stir the composition for at least 1, 2, 3, 4, 5, 6, 7, 8, 9, or 10 minutes, such as for about 5 minutes.

A useful parameter for referring to dUSPIO compositions is by their wt.-% iron. Iron content can be determined using techniques known in the art, such as inductively coupled plasma mass spectrometry (ICP-MS), atomic emission spectroscopy (AES), and atomic absorption spectroscopy (AAS); AAS and ICP-MS are preferred techniques, AAS is most preferred. In preferred embodiments, the invention provides the method according to the invention, wherein the ultrafiltrated composition of step i) or the adjusted composition of step iii) comprises from about 1.5-2.5 wt.-% iron, preferably from about 1.6-2.3 wt.-% iron, more preferably from about 1.7-2.1 wt.-% iron, most preferably from about 1.8-2.0 wt.-% iron, such as 1.9 wt.-% iron.

In preferred embodiments of the method according to the invention, the ultrafiltrated composition of step i) can further comprise a pharmaceutically acceptable excipient. This excipient can be present in the ultrafiltrated composition as provided for use in step ii), or it can be present prior to ultrafiltration, or it can be present in solvents or diluents or washing fluids as used during step i). Examples of pharmaceutically acceptable excipients are provided later herein. For step i), a preferred pharmaceutically acceptable excipient is water such as water for injection, milliQ water, or SuperQ water, more preferably water for injection. Throughout this application, reference to water as a solvent or excipient is intended to also encompass reference to pharmaceutically acceptable aqueous solutions such as 0.9 wt.-% NaCl solution in water, or phosphate buffered saline (PBS) solutions.

ii) Analysis of the Unadjusted Nanoparticles

In step ii) the amount of dextran-T10 present in the ultrafiltrated composition of step i) is determined. It is preferable to store the ultrafiltrated composition at 2-8° C. during this determination. It is preferable to stir the ultrafiltrated composition for at least about 1, 2, 3, 4, 5, 6, 7, 8, 9, or 10 minutes, such as for about 5 minutes, prior to this determination. The determination of dextran-T10 can be performed by standard chromatographic techniques such as HPLC and GPC, via UV-VIS spectrophotometry for example using colorimetric assays, or via tests for total organic carbon (TOC), preferably using a TOC analyzer such as a Shimadzu TOC-L analyzer. A preferred colorimetric assay is that of Dubois et al. (1956, Anal. Chem., 28:350-356), as used by Jung (1995), wherein reagents such as phenol and sulfuric acid are used to produce a quantifiable amount color proportional to the total amount of dextran-T10. It is preferable to perform this assay when dextran-T10 is the only carbohydrate present in the composition because the colorimetric assay determines the total carbohydrate content and is not specific for dextran-T10.

In preferred embodiments, step ii) further comprises determining the amount of iron in the ultrafiltrated composition of step i). Iron content can be determined using techniques known in the art, such as inductively coupled plasma mass spectrometry (ICP-MS), atomic emission spectroscopy (AES), and atomic absorption spectroscopy (AAS); AAS is a preferred technique.

Preferably, the amount of dextran-T10 as determined is expressed as wt.-% dextran-T10 relative to the wt.-% of iron in the composition. Preferably, the amount of iron in the composition is expressed as the wt.-% of iron in the composition.

iii) Adjusting the Ultrafiltrated Composition

In step iii) the ultrafiltrated composition is adjusted by adding an amount of dextran-T10 to the ultrafiltrated composition of step i). The adjusted composition comprises 140-160 wt.-% of dextran-T10 relative to the wt.-% of iron. Preparation of dUSPIO generally involves ultrafiltration, during which dextran-T10 is slowly dissociated from the dUSPIO. This is because the association of dextran-T10 with the iron oxide core is not covalent (Jung 1995), and exists in an equilibrium. Temporarily dissociated dextran-T10 can be washed away during ultrafiltration, before it can associate with the dUSPIO again. As a result, ultrafiltration reduces the amount of dextran in dUSPIO. Thorough ultrafiltration is required to obtain dUSPIOs that are sufficiently pure to be suitable for their use, which generally requires good manufacturing processes to be used, which should be very reproducible. The loss of dextran-T10 during ultrafiltration cannot be controlled or mitigated during ultrafiltration. Accordingly, dUSPIO known in the art have dextran-T10 contents that have a dispersion of over 15%. A dispersion of at most 15%, preferably at most 10% is desired.

In this context, dispersion is preferably defined as 3 times the standard deviation between a plurality of production batches of dUSPIO divided by the mean value, expressed as a percentage, more preferably rounded up to the nearest full percentage. This plurality preferably comprises at least 2, 3, 4, 5, 6, 7, 8, 9, or more production batches of dUSPIO, more preferably at least 3, 4, 5, or 6 production batches, even more preferably at least 4, 5, or 6, most preferably at least 6 production batches. A composition of dUSPIO is said to be specified at a certain percentage when its dispersion is below that percentage.

The inventors surprisingly found that adjustment of the amount of dextran-T10 after ultrafiltration, so that the adjusted composition comprises 140-160 wt.-% of dextran-T10 relative to the wt.-% of iron, leads to adjusted compositions that can be further filtered or manipulated while maintaining a reliable amount of dextran-T10, with a dispersion of at most 15%, more preferably of at most 10%. In preferred embodiments, the dUSPIO obtained by a method according to the invention has a dispersion of less than 14, 13, 12, 11, 10, 9, 8, 7, 6, or 5%. Determination of dextran content has been described elsewhere herein. In more preferred embodiments, adjustment of the amount of dextran-T10 after ultrafiltration is such that the adjusted composition comprises 140-158 wt.-%, more preferably 140-155 wt.-%, even more preferably 145-155 wt.-% of dextran-T10 relative to the wt.-% of iron.

As will be clear to a skilled person, content ranges are ranges that are actively sought during the preparation method. Therefore the adjusted composition comprising 140-160 wt.-% of dextran-T10 relative to the wt.-% of iron does not comprise less dextran-T10 than 140 wt.-%, and does not comprise more dextran-T10 than 160 wt.-%. Accordingly in preferred embodiments a composition specified to comprise a range of a particular component does not comprise less of that component than specified as the lower limit of the indicated range, and does not comprise more of that component than specified as the upper limit of the indicated range. As a highly preferred example, an adjusted composition comprising 140-160 wt.-% of dextran-T10 relative to the wt.-% of iron comprises at least 140 wt.-% and at most 160 wt.-% of dextran-T10 relative to the wt.-% of iron.

As used herein, adjustment preferably refers to addition of an amount that is substantially the difference between a determined value and a target value. Accordingly, adjustment of dextran-T10 is preferably the addition of such an amount of dextran T-10 to a composition that the adjusted composition comprises a desired total amount of dextran T-10. As a non-limiting example, if for a composition the amount of dextran-T10 in step ii) is found to be 120 wt.-% relative to the wt.-% of iron, and production of a composition comprising 140 wt.-% of dextran is intended, then adjustment would entail the addition of 20 wt.-% of dextran-T10 to the composition.

In step iii), further substances can be added to the ultrafiltrated composition or to the adjusted composition. Suitable further substances are dextrans that are not dextran-T10, tonicity agents, diluents, and pharmaceutically acceptable excipients as later defined herein. Accordingly, in preferred embodiments is provided the method according to the invention, wherein the ultrafiltrated composition of step i) further comprises a pharmaceutically acceptable excipient, or wherein the adjusted composition of step iii) further comprises a pharmaceutically acceptable excipient, wherein the pharmaceutically acceptable excipient is preferably added before or after adjustment of the dextran-T10 content, more preferably after adjustment. Suitable pharmaceutical excipients are known in the art, examples are Further preferred excipients are adjuvants, binders, desiccants, anti-caking agents, dyes, diluents, and tonicity agents such as described below.

Accordingly, in preferred embodiments is provided the method according to the invention, wherein step iii) further comprises adding an amount of a further dextran such as dextran-T1 to the ultrafiltrated composition or to the adjusted composition. This further dextran is not dextran-T10; suitable further dextrans are dextran-T1, dextran-T5, dextran-T20, and dextran-T40, and dextran-T1 is the most preferred further dextran because it can help reduce any possible immune response to dextran in general. The further dextran can be added so that the adjusted composition comprises 10-300 wt.-% of the further dextran, preferably 50-250 wt.-%, more preferably 100-200 wt.-%, even more preferably 140-160 wt.-%, most preferably 145-155 wt.-% such as 150 wt.-%, wherein the wt.-% is relative to the wt.-% of iron. Preferably, the further dextran is added after or during adjustment, most preferably during adjustment—in other words, the further dextran is preferably added simultaneously with the dextran-T10 used for adjustment. Accordingly, in a preferred embodiment is provided the method according to the invention, wherein step iii) further comprises adding an amount of a further dextran such as dextran-T1 to the ultrafiltrated composition or to the adjusted composition, preferably to the adjusted composition, so that the adjusted composition comprises 140-160 wt.-% of the further dextran such as dextran-T1 relative to the wt.-% of iron. The inventors have found that it is advantageous to have substantially equal amounts by weight of dextran-T1 and dextran-T10 in a composition according to the invention. In preferred embodiments, in step iii) an amount of dextran-T1 is added to the adjusted composition so that it comprises substantially equal amounts by weight of dextran-T1 and dextran-T10. The invention thus provides the method according to the invention, wherein the adjusted composition comprises a substantially equal amount by weight of dextran-T10 and of dextran-T1. It is to be understood that reference is made to total dextran contents, considering both bound and free dextran.

Accordingly, in preferred embodiments is provided the method according to the invention, wherein step iii) further comprises adding a tonicity agent such as a citrate to the ultrafiltrated composition or to the adjusted composition, preferably in an amount of 7-12 wt.-% relative to the wt.-% of iron. More preferably, the tonicity agent is present in about 7.5-11.5 wt.-%, 8-11 wt.-%, 8.5-10.5 wt.-%, or 9-10 wt.-%, even more preferably 8.5-10.5 wt.-% or 9-10 wt.-%, most preferably 9-10 wt.-% such as 9.5 wt.-%. In the context of this invention, a tonicity agent reduces local irritation by preventing osmotic shock at the site of application of a composition. Tonicity agents are known in the art. Suitable tonicity agents are hexoses such as dextrose, amino acids such as glycerin, sugar alcohols such as mannitol, alkali halide salts such as potassium chloride and sodium chloride, and weak organic acid salts such as citrates and ethylenediaminetetraacetic acid (EDTA) salts. Weak organic acid salts are preferred because they increase stability of the obtained dUSPIO and have a good effect on their zeta potential; for this, citrates are particularly preferred. Suitable citrates are citric acid trisodium salt dihydrate, citric acid disodium salt, and citric acid sodium salt, a most preferred citrate is citric acid trisodium salt dihydrate.

In some embodiments of the invention, the method according to the invention further comprises the step of lyophilizing the adjusted composition. Lyophilisation is known in the art. This lyophilisation can be performed on the adjusted composition either before or after its filtering in step iv). The resulting lyophilisate is a suitable form for storage of the adjusted composition. For lyophilisation the adjusted composition is preferably snap frozen in a vial. Preferably, nitrogen is used during lyophilisation to break the vacuum in the lyophilisator, or in the head-space of the freeze-dried vials, or in both. When a method according to the invention comprises a lyophilisation step, it is preferred that both dextran-T1 and a tonicity agent such as citrate are added to the adjusted composition as described above, prior to the lyophilisation step. The combination was surprisingly found to improve lyophilisation results.

iv) Filtering the Adjusted Composition

Step iv is a filtering step, which is useful to clear aggregates from dUSPIO compositions. A preferred filtration in this context is filtration through a 0.05-0.5 μm filter, preferably a 0.1-0.3 μm filter such as a 0.2 μm filter. As discussed above, filtration can separate dextran from the iron oxide cores in the dUSPIO, altering the exact contents of the composition. The adjusted compositions as produced with a method according to the invention are more robust to such changes, because the adjustment neutralizes the influence that previous filtration steps have had. As such, a filtered adjusted composition is of more certain contents, and thus more reliable than a filtered composition that has not been adjusted. The invention provides the method according to the invention, further comprising the step of lyophilizing the adjusted composition after filtration.

### Composition

In another aspect, the invention provides a composition comprising:

i) ultra-small superparamagnetic iron oxide nanoparticles coated with dextran-T10;

ii) free dextran-T10;

iii) optionally a tonicity agent such as a citrate;

wherein the composition comprises 140-160 wt.-% of dextran-T10 relative to the wt.-% of iron. Such a composition is referred to herein as a composition according to the invention. Preferably it is a pharmaceutical composition. The dextran-T10 of which 140-160 wt.-% is comprised relative to the wt.-% of iron is the total sum of dextran-T10, that is both the dextran-T10 covering the dUSPIO of i) as well as the free dextran-T10 of ii). Preferably, a composition according to the invention is obtainable by a method according to the invention, more preferably directly obtained by a method according to the invention. Accordingly, compositions as described in the section above are within this aspect of the invention.

In preferred embodiments, the composition comprises further pharmaceutically acceptable excipients as defined elsewhere herein. In this context, a further dextran such as dextran-T1 is preferred, and a tonicity agent such as citrate is also preferred.

In preferred embodiments within this aspect is provided the composition according to the invention, wherein the concentration of dextran-T10 has a dispersion of at most ±15%, ±14%, ±13%, ±12%, ±11%, ±10%, ±9%, or below, preferably of at most ±10%, more preferably of at most ±9%. In this context, dispersion is preferably defined as 3 times the standard deviation between a plurality of production batches of composition according to the invention, more preferably rounded up to the nearest full percentage. This plurality preferably comprises at least 2, 3, 4, 5, 6, 7, 8, 9, or more production batches of composition according to the invention, more preferably at least 4, 5, or 6 production batches, most preferably at least 6 production batches. A composition according to the invention is said to be specified at a certain percentage when its dispersion is below that percentage. Accordingly, the preferred embodiments provide the composition according to the invention which is specified for dextran-T10 at 10%.

In a particular embodiment, the invention provides a plurality of compositions according to the invention, preferably wherein each composition is from a different production batch, wherein the concentration of dextran-T10 has a dispersion of at most ±15%, ±14%, ±13%, ±12%, ±11%, ±10%, or below, preferably of at most ±10% between compositions. This plurality preferably comprises at least 2, 3, 4, 5, 6, 7, 8, 9, or more compositions according to the invention, more preferably at least 3, even more preferably at least 4, 5, or 6 compositions, most preferably at least 6 compositions. This plurality preferably comprises at most 100000 compositions. Preferably, at least two compositions from the plurality are from a different production batch. Preferably, each composition has substantially the same contents, for example regarding excipients. More preferably, each composition is substantially identical outside of the variations in exact ingredient content that occur when a production method is repeated more than once, for example each composition is identical outside of the dispersion that occurs for constituent components such as dextran-T10. In preferred embodiments, the plurality is specified for dextran-T10 at 15%, 14%, 13%, 12%, 11%, or 10%, most preferably at 10%.

Compositions according to the invention are of high stability. A useful parameter for assessing this stability is the unimodal mean diameter of the nanoparticles comprised in the compositions according to the invention. If nanoparticles aggregate, the unimodal mean diameter will increase. If nanoparticles dissolve or dissociate or otherwise degrade, the unimodal mean diameter will decrease. A stable unimodal mean diameter reflects a stable population of nanoparticles, and thus a stable composition according to the invention. In embodiments of this aspect is provided the composition according to the invention, wherein the unimodal mean diameter of the nanoparticles is stable for at least 1, 2, 3, 4, 5, 6, 7, 8, 9, 10, 11, or 12 months, or longer. Preferably, the unimodal mean diameter is stable for at least 4, 5, 6 months, or longer, most preferably for at least 6 months or longer. In preferred embodiments, the stability is at about room temperature, such as at 20-30° C., such as at 25° C.

In this context, a stable mean unimodal diameter is a mean unimodal diameter that does not deviate from a value that was measured, at a different time point for the same composition, by more than 6%, 7%, 8%, 9%, 10%, 11%, 12%, 13%, 14%, 15%, or more, preferably by more than 6%, 7%, 8%, 9%, 10%, or more, more preferably by more than 6%.

Compositions according to the invention are preferably lyophilisates. In other embodiments, the compositions according to the invention are reconstituted lyophilisates. Suitable reconstitution fluids are water for injection, preferably comprising pharmaceutically acceptable salts such as 0.9 wt.-% sodium chloride. Reconstituted compositions according to the invention preferably comprise from about 1.5-2.5 wt.-% iron, preferably from about 1.6-2.3 wt.-% iron, more preferably from about 1.7-2.1 wt.-% iron, most preferably from about 1.8-2.0 wt.-% iron, such as 1.9 wt.-% iron.

In preferred embodiments, the composition is provided as a lyophilisate and water for injection both in separate containers. A suitable container for such a lyophilisate is a glass vial such as a clear glass vial, for example a clear type I glass vial. It is preferably closed with a rubber stopper such as a bromobutyl or chlorobutyl rubber stopper and preferably sealed, such as with an aluminum crimp-on seal.

The following are preferred embodiments of compositions according to the invention:

1. A composition according to the invention comprising:

i) ultra-small superparamagnetic iron oxide nanoparticles coated with dextran-T10;

ii) free dextran-T10;

wherein the composition comprises about 19 mg/g iron and about 28.5 mg/g dextran-T10.

1′. The composition of embodiment 1, not comprising any further iron or dextran-T10.
2. The composition of embodiment 1 or 1′, further comprising about 28.5 mg/g Dextran-T1.
3. The composition of any of embodiments 1-2, further comprising about 2.45 mg/g sodium citrate.
4. The composition of any of embodiments 1-3, wherein the remainder substantially consists of water such as water for injection.
5. The composition of any of embodiments 1-3, wherein the remainder substantially consists of a 0.9 wt.-% NaCl solution in water.
6. The composition of any one of embodiments 1-5, substantially not comprising any further substances or ingredients.
7. A composition according to the invention comprising:

i) ultra-small superparamagnetic iron oxide nanoparticles coated with dextran-T10;

ii) free dextran-T10;

wherein the composition comprises about 210.4 mg iron and about 315.5 mg dextran-T10, preferably not comprising any further iron or dextran-T10.

8. The composition of embodiment 7, further comprising about 315.5 mg dextran-T1.
9. The composition of embodiment 7 or 8, further comprising about 27.2 mg sodium citrate.
10. The composition of any one embodiments 7-9, substantially not comprising any further substances or ingredients.
11. The composition of any one embodiments 7-10, packaged in a vial such as a glass vial.
12. The composition of embodiment 11, wherein the vial comprises an inert atmosphere such as a nitrogen atmosphere.
13. The composition of any one embodiments 7-12, in combination with a separate container of water such as water for injection.
14. The composition of any one embodiments 7-12, in combination with a separate container of a 0.9 wt.-% NaCl solution in water.
1a. A composition according to the invention comprising:

i) ultra-small superparamagnetic iron oxide nanoparticles coated with dextran-T10;

ii) free dextran-T10;

wherein the composition comprises about 17-21 mg/g iron and about 26-31 mg/g dextran-T10, preferably not comprising ant further iron or dextran-T10.

2a. The composition of embodiment 1a, further comprising about 26-31 mg/g Dextran-T1.
3a. The composition of embodiment 1a or 2a, further comprising about 2.2-2.7 mg/g sodium citrate.
4a. The composition of any of embodiments 1a-3a, wherein the remainder substantially consists of water such as water for injection.
5a. The composition of any of embodiments 1a-3a, wherein the remainder substantially consists of a pharmaceutically acceptable salt solution such as 0.9 wt.-% NaCl solution in water.
6a. The composition of any one of embodiments 1a-5a, substantially not comprising any further substances or ingredients.
7a. A composition according to the invention comprising:

i) ultra-small superparamagnetic iron oxide nanoparticles coated with dextran-T10;

ii) free dextran-T10;

wherein the composition comprises about 190-230 mg iron and about 285-345 mg dextran-T10, preferably not comprising ant further iron or dextran-T10.

8a. The composition of embodiment 7a, further comprising about 285-345 mg dextran-T1.
9a. The composition of embodiment 7a or 8a, further comprising about 25-30 mg sodium citrate.
10a. The composition of any one embodiments 7a-9a, substantially not comprising any further substances or ingredients.
11a. The composition of any one embodiments 7a-10a, or 15a, or 16a, packaged in a vial such as a glass vial.
12a. The composition of embodiment 11a, wherein the vial comprises an inert atmosphere such as a nitrogen atmosphere.
13a. The composition of any one embodiments 7a-12a, in combination with a separate container of water such as water for injection.
14a. The composition of any one embodiments 7a-12a, in combination with a separate container of a pharmaceutically acceptable salt solution such as a 0.9 wt.-% NaCl solution in water.
15a. The composition of any one embodiments 7a-10a, combined with further composition of any one embodiments 7a-10a in a single composition.
16a. The composition of any one embodiments 7a-10a, wherein a volume of the composition has been removed from the composition.
17a. The composition of any one of embodiments 1a-6a, combined with further composition of any one embodiments 1a-6a in a single composition.

18a. The composition of any one embodiments 1a-6a, wherein a volume of the composition has been removed from the composition.

### Use

The composition according to the invention is a useful MRI contrast agent. Accordingly, the invention provides the composition according to the invention, for use as a medicament, wherein the medicament if preferably for in vivo diagnostics, more preferably for use as an MRI contrast agent. This is referred to hereinafter as a composition for use according to the invention.

In a particular embodiment, the composition for use according to the invention is for use in iron replacement therapy, for example to treat anemia.

In a particular embodiment, the composition for use according to the invention is for use in the diagnosis of multiple sclerosis. The composition for use according to the invention allows for identification of active lesions in the brain, differentiating them from old, non-active lesions.

In a particular embodiment, the composition for use according to the invention is for use in the diagnosis of atherosclerosis. The composition for use according to the invention allows for identification of active atheromatous plaques in arteries, allowing determination of plaques prone to rupture. As such, the composition for use according to the invention can help prevent thrombo-embolic complications.

In a particular embodiment, the composition for use according to the invention is for use in angiography.

The MRI contrast agent is preferably for visualization of the reticuloendothelial system, liver, spleen, lymph nodes, bone marrow, atherosclerosis, and active lesions, more preferably for visualization of lymph nodes, even more preferably for visualization of liver, spleen, lymph nodes, bone marrow, atherosclerosis, and active lesions, most preferably for visualization of lymph nodes. In particular embodiments, the in vivo diagnostics is for diagnosis of cancer, more preferably for diagnosis for a cancer selected from the group consisting of solid cancers like prostate, bladder, breast, or gynecological cancers. A preferred type of diagnosis is the lymph node staging or characterization in MRI. For example, lymph nodes can be determined to be tumorous or non-tumorous. The composition for use according to the invention is preferably a powder such as a lyophilisate, or a concentrate, or a solution for parenteral administration such as infusion, preferably slow infusion. Administration is preferably at a posology of at least 0.5, 1, 1.5, 2, 2.5, 3, 3.5, 4, 4.5, or 5 mg iron per kg body weight, more preferably of at least 1.5, 2, 2.5, 3, or 3.5 mg iron per kg body weight. Administration is preferably at a posology of at most 1, 1.5, 2, 2.5, 3, 3.5, 4, 4.5, 5, or 5.5 mg iron per kg body weight, more preferably of at most 3, 3.5, or 4 mg iron per kg body weight. A preferred posology is 1-4 mg iron per kg body weight, more preferably 2-3 mg iron per kg body weight, such as 2.6 mg iron per kg body weight.

After reconstitution of the powder or concentrate it gives a solution of superparamagnetic iron oxide nanoparticles stabilised with dextran, preferably also with a tonicity agent such as sodium citrate. Reconstitution is preferably with a suitable pharmaceutical dilution fluid, such as water for injection, preferably comprising pharmaceutically acceptable salts such as 0.9 wt.-% sodium chloride. The active substance consists of iron oxide cores of nanometric size which provide it with properties of contrast in relaxation imaging of protons, and further consists of stabilizers (dextran-T10, preferably also dextran-T1, and preferably a tonicity agent such as sodium citrate) which ensure proper dispersion and stabilization of these cores.

The composition for use according to the invention is useful for Medical Resonance Imaging (MRI), and belongs to the known group of contrast agents specific to the reticuloendothelial system (liver, spleen, lymph nodes, bone marrow) and particularly to the sub-group known as Ultra-Small Particles of Iron Oxide (USPIO) whose particle diameter are below 50 nm. The composition for use according to the invention comprises nanoparticles of small size (mean diameter, preferably laser light scattering mean diameter, between 20 and 50 nm, preferably below 35 nm, more preferably 25-35 nm, such as about 30 nm) which give its specific and preferential targeting for lymph nodes.

Formulation of medicaments, ways of administration, and the use of pharmaceutically acceptable excipients are known and customary in the art and for instance described in Remington;

The Science and Practice of Pharmacy, 21st Edition 2005, University of Sciences in Philadelphia.

### GENERAL DEFINITIONS

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value more or less 1% of the value.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a substance as a medicament as described in this document can also be interpreted as the use of said substance in the manufacture of a medicament. Similarly, whenever a substance is used for treatment or as a medicament, it can also be used for the manufacture of a medicament for such treatment. Products for use as described herein are suitable for use in methods of treatment or of diagnosis as described herein.

The present invention has been described above with reference to a number of exemplary embodiments. Modifications and alternative implementations of some parts or elements are possible, and are included in the scope of protection as defined in the appended claims. All citations of literature and patent documents are hereby incorporated by reference.

### DESCRIPTION OF DRAWINGS

FIG. 1—Sketch of an iron oxide core stabilised by dextran. Parts are not to scale.

FIG. 2—Flowchart depicting a method according to the invention. The steps with a bold dashed outline are the steps wherein an adjusted nanoparticle composition is produced.

FIG. 3—Analysis of different dextran fractions after ultracentrifugation of an adjusted nanoparticle composition. Dextran-T1 is revealed to remain substantially unassociated with the nanoparticles, whereas about 70% of Dextran-T10 is associated with the nanoparticles.

FIG. 4—Analysis of the influence of Dextran-T1 or of citrate on different parameters of the adjusted nanoparticle composition. A) influence of various concentrations of Dextran-T1 and citrate on pH; no clear optimum can be determined. B) influence of various concentrations of Dextran-T1 and citrate on unimodal diameter; an optimal zone can be found when Dextran-T1 is at 20 to 30 mg/g while citrate is beneath 2.6 mg/g. C) influence of various concentrations of Dextran-T1 and citrate on nanoparticle-bound citrate levels; dextran-T1 has no significant effect on this parameter, and citrate in excess of about 2.1 mg/g has no more significant effect.

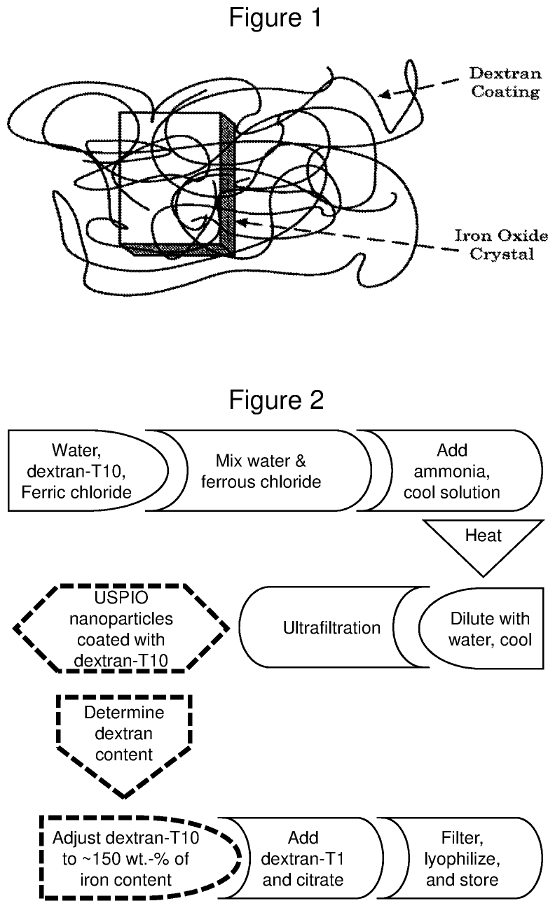

### EXAMPLES

### Example 1—Production of Adjusted dUSPIO

### Provision of Ultrafiltrated dUSPIO (First Seven Shapes in FIG. 2)

The description of the manufacturing process given hereafter is for a clinical batch size (143 g iron).

Step 1: Preparation of Dextran-Iron solution—Purified water (1950±40 g) is added into a 5 L container labeled A. While mixing the purified water, Dextran-T10 (1143 g) is added to the container. Ferric chloride (84.7±1.7 g) is added to the Dextran solution and the solution is mixed for a minimum of 3 hours. Transfer the Dextran-Iron solution to container “B” through a 0.2μ filter and a 0.45 μm prefilter, then transfer into the reactor using a peristaltic pump. The Dextran-Iron solution is cooled under nitrogen with stirring in the reactor until the temperature reaches 1-6° C.

Step 2: Preparation of Ferrous Chloride Solution—Purified water (46.8±0.9 g) is added into the 250 mL container labelled C. The purified water is stirred while 34.8 g±0.7 g of ferrous chloride tetrahydrate is added. The stirring is continued for a minimum of 110 minutes. The solution is stored under nitrogen at 2-8° C. until needed. Step 3: Preparation of Strong Ammonia solution—Strong ammonia solution (103.2±2.0 g) is weighed in the fume hood into a 250 mL container labelled D and cooled to 2-8° C.

Step 4: Heating to 80° C.±5° C.—The reactor contents are stirred under nitrogen while the ferrous chloride solution is pumped through a 0.2μ filter into the reactor. The ammonium hydroxide solution is then added to the reactor. The reactor is heated and stirred until the contents reach 80° C.±5° C.

Step 5: Dilution with purified water—8660±150 mL of 80° C.±5° C. purified water is then added to the reactor with continued stirring. The temperature of the reactor is cooled to 25° C.±5° C. The solution is filtered via a 0.2 μm filter (and a 0.45 μm prefilter) into container F.

Step 6: Ultrafiltration—Six superparamagnetic iron oxide batches are filtered through a 0.2μ filter (and a 0.45 μm prefilter) directly into the tank of the ultrafilter apparatus fitted with a 100,000 Dalton cut-off cartridge membrane. A constant flow ultrafiltration procedure is used for purification. The solution is processed through the ultrafilter until the retained volume is 25.5 L. The effluent is discarded during all the ultrafiltration steps. The iron oxide solution is processed through the ultrafilter again by washing the solution with 51 L water for injection while keeping the volume at 25.5 L (continuous ultrafiltration). The solution is processed through the ultrafilter until the retained volume is 9 L. The iron oxide solution is processed through the ultrafilter again by washing the solution with 24 L water for injection while keeping the volume at 9 L. A minimum of two more cycles of ultrafiltration is performed. The conductivity of the effluent is tested. If it is less than 25 μS, the ultrafiltration is continued to a volume of 2.16±0.1 L and the ultrafiltration is complete. If the conductivity is greater than 25 μS, either one or two additional ultrafiltration cycles are performed with conductivity testing after each cycle. Both conditions must be satisfied for ultrafiltration to be complete:
First, a minimum of 10 diafiltration volumes of water for injection must have been used,
Second, a conductivity of less than 25 μS must be reached. The solution is removed from the ultrafilter tank and filtered via a 0.2 μm filter (with 0.45 μm prefilter) into a 10 L container. Water for injection (1440±40 mL) is added into the ultrafilter tank to rinse it and then added to the 10 L container.

### Analysis of Ultrafiltrated dUSPIO (Shape 8 in FIG. 2)

Step 7—The ultrafiltrated solution is stirred for a minimum of 5 minutes and then analysed for total iron concentration and dextran content using Atomic Adsorption Spectroscopy for determination of iron content and TOC (total organic carbon) for total dextran content. The solution is stored at 2° C.-8° C. until the results are available.

### Adjustment of dUSPIO and Further Processing (Shapes 9, 10, part of 11 in FIG. 2)

Step 8: Preparation of Dextran-T10, Dextran-T1 and sodium citrate dihydrate solution—The amount of a Dextran-T10, Dextran-T1 and sodium citrate dihydrate solution in water for injection is calculated based on the iron concentration and dextran-T10 concentration from step 7 such that in the final formulation the concentration of iron is 18-20 mg/g, the total concentration of dextran is 53-61 mg/g, the ratio of dextran-T10 and dextran-T1 is 1/1 g/g and the citrate content is 1.5-2.1 mg/g. Water for injection is added into a 10 L container. The water for injection is stirred, and Dextran-T10, Dextran-T1, and sodium citrate dihydrate are added (good results were obtained with slow addition). The solution is stirred for a minimum of 15 minutes.

Step 9: Addition of Dextran-T10, Dextran-T1 and sodium citrate dihydrate Solution—The solution of step 7 is mixed and the final formulation is prepared by adding the solution of step 8 through a 0.2 μm filter and a 0.45 μm prefilter while continuously mixing.

Step 10: filtration—The solution is filtered through a 0.2μ filter into 10 L containers for storage and shipping. This step can be repeated if necessary (e.g. in case of filter failure).

Table 1 shows examples of adjustment of three different batches that were prepared according to steps 1-6 above.

TABLE 1
examples of the preparation of adjusted batches

| Action | Batch 1 | Batch 2 | Batch 3 |
|---|---|---|---|
| Conductivity final ultrafiltration | 6 | 17 | 9 |
| flowthrough (μS/cm) | | | |
| Total weight of batch (g) | 2542 | 3582 | 3730 |
| Fe concentration (mg/g) | 41.0 | 42 | 40.5 |
| Dextran-T10 content (mg/g) | 31 | 33 | 31 |
| Total Dextran-T10 after | 78.8 | 118.2 | 115.6 |
| ultrafiltration (g) | | | |
| Dextran-T10 added for | 77.7 | 107.4 | 110.9 |
| adjustment (g) | | | |

These compositions were further treated as follows:

| | | | |
|---|---|---|---|
| Dextran-T1 added (g) | 156.1 | 225.5 | 226.6 |

### Example 2—Comparison of Adjusted dUSPIO to Unadjusted dUSPIO

The adjusted composition of example 1 is an optimised formula because it is adjustment for dextran-T10 during the manufacture. This adjustment allows to ensure a better reproducibility of the concentration of dextran in the finished product (±10%) and increases stability of the unimodal mean diameter. To demonstrate that the formulae are otherwise equivalent, a comparison study was performed between adjusted (entire protocol of example 1) and unadjusted (steps 1-6 of example 1) compositions. Both compositions comprised 1.8 mg/g citrate ion and 28.5 mg of dextran-T1 and were stabilised as a lyophilizate and stored at 4° C. and 25° C. for 6 months. Zeta potential was measured using a Malvern Zetasizer. Unimodal mean diameter was measured using Laser Light Scattering (suitable models are Malvern Zetasizer, Brookhaven BI 90, and Malvern 4700).

TABLE 2
comparison of adjusted and unadjusted dUSPIO

| Unadjusted | | | | | Adjusted | | | |
|---|---|---|---|---|---|---|---|---|
| 0 | 1 | 3 | 6 | Months | 0 | 1 | 3 | 6 |
| 8.2 | 8.2 | 8.2 | 8.1 | pH 4° C. | 8.0 | 8.1 | 8.1 | 8.1 |
| 8.2 | 8.1 | 8.1 | 8.1 | pH 25° C. | 8.0 | 8.1 | 8.1 | 8.1 |
| 36 | 36 | 39 | 37 | Unimodal mean | 36 | 36 | 36 | 38 |
| | | | | diameter 4° C. | | | | |
| 36 | 36 | 40 | 25 | Unimodal mean | 36 | 36 | 38 | 36 |
| | | | | diameter 25° C. | | | | |
| | | | −47.4 | Zeta potential | | | | −51.4 |
| | | | | water | | | | |
| | | | −19.9 | Zeta potential | | | | −21.3 |
| | | | | 0.9% NaCl | | | | |

For both compositions, at 4 and 25° C., the pH does not vary in time, and the zeta potential is highly similar in both water or salt solution. For the unadjusted composition the unimodal mean diameter varied more than 7% at 4° C., and more than 35% at 25° C., while under both conditions the adjusted composition remained at about 5% variation.

Unadjusted and adjusted batches were also analysed for their total dextran content. Adjusted compositions could be specified at ±10% having a dispersion of ±9%. Unadjusted compositions had a dispersion of ±17%.

TABLE 3
comparison of dextran concentrations

| Unadjusted | | Adjusted |
|---|---|---|
| Total dextran | | Total dextran |
| (mg/g) | Batch No. | (mg/g) |
| 50 | 1 | 56 |
| 51 | 2 | 54 |
| 53 | 3 | 54 |
| 49 | 4 | 54 |
| 48 | 5 | 58 |
| 45 | 6 | 55 |
| 49 | Mean | 55 |
| 8.2 | 3x σ | 4.8 |
| ±17% | Dispersion | ±9% |

### Example 3—Analysis of dUSPIO

### Analysis of Free and Associated Dextran Fractions

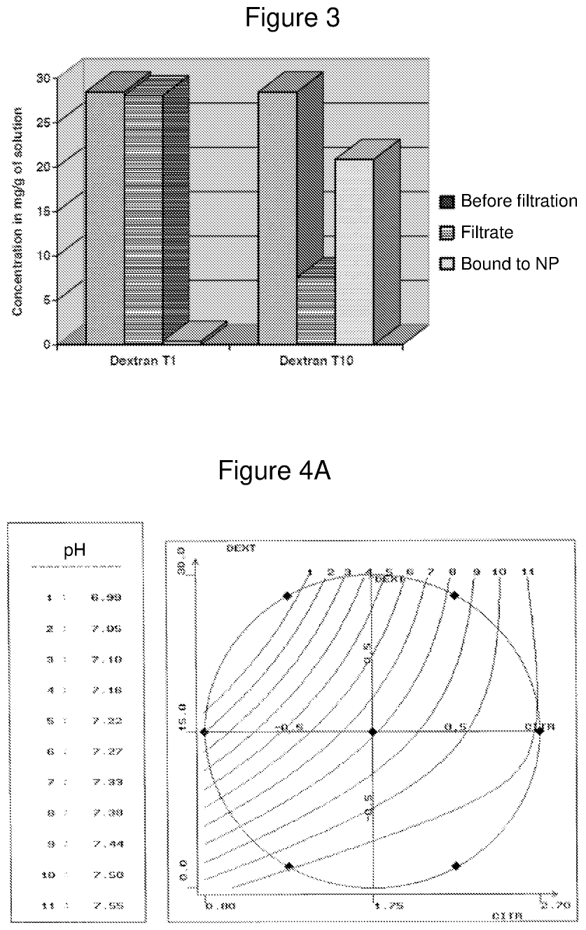

Dextran fractions of the composition as prepared in example 1 were determined after a single step of ultracentrifugation at 30 kDa MWCO as described above for the ultrafiltrated composition, after having allowed the composition time to equilibrate. Dextran was identified using gel permeation chromatography, results are shown in FIG. 3. Dextran-T1 is revealed to remain substantially unassociated with the nanoparticles, whereas about 70% of Dextran-T10 is associated with the nanoparticles. Dextran-T1 remains at the free state in the solvent and does not alter the dextran-T10/iron oxide interaction. The data also confirm that the particle-bound fraction of dextran-T10 represents 70% of the total quantity of dextran-T10 when the composition is in equilibrium.

### Determination of Optimal Excipient Concentrations

Different formulations were prepared as shown in Table 4, to be used for analysis according to a Doelhert's matrix (see for example Sautour et al., J. App. Microbiol., 2001, 91, 900-906). Each formula was stabilised as a lyophilizate, and was analysed after reconstitution in 0.9% NaCl. Each formula was stored at room temperature and at 55° C. for 12 months. The following parameters were analysed: pH, particle size by laser scattering, zeta potential, and bound citrate. Bound citrate can be assessed by HPLC techniques, or LCMS, on supernatant after reconstitution. The results are analysed using Nemrod 3.0 software.

The factors (concentration in citrate and concentration in dextran-T1) are symbolised by X1 et X2, respectively. The function that models the Y responses (parameters) measured in terms of X1 and X2 is:

Y=b₀+b₁X₁+b₂X₂+b₁₁X₁²+b₂₂X₂²+b₁₂X₁X₂

TABLE 4
compositions as used in this example

| Composition | | | | | Unimodal diameter | | | Bound citrate |
|---|---|---|---|---|---|---|---|---|
| Citrate | Dextran-T1 | pH | | | T = 0 | 25° C. | 55° C. | 25° C. |
| (mg/g) | (mg/g) | T = 0 | 25° C. | 55° C. | (nm) | (nm) | (nm) | (mg/g) |
| 0.77 | 14.4 | 7.87 | 7.88 | 6.99 | 36 | 46 | 59 | 0.69 |
| 1.22 | 1.9 | 7.99 | 8.05 | 7.60 | 53 | 101 | 157 | 0.91 |
| 1.22 | 26.8 | 8.07 | 8.03 | 7.01 | 30 | 30 | 31 | 0.90 |
| 1.67 | 14.4 | 8.09 | 7.97 | 7.37 | 32 | 25 | 38 | 1.09 |
| 1.67 | 14.4 | 8.10 | 7.98 | 7.38 | 32 | 34 | 38 | 1.08 |
| 2.13 | 1.9 | 8.15 | 8.21 | 7.56 | 42 | 68 | 107 | 1.16 |
| 2.13 | 26.8 | 8.10 | 8.10 | 7.35 | 32 | 32 | 34 | 1.12 |
| 2.58 | 14.4 | 8.19 | 8.11 | 7.61 | 34 | 37 | 41 | 1.19 |
| 1.81 | 28.7 | 8.08 | 8.04 | 7.35 | 28 | 30 | 31 | 1.12 |

### pH

In any experiment performed, no variation of the pH at 25° C. is observed. This parameter is not relevant in this study. The response of pH (55° C.) is modelled by the following polynomial:

pH=7.38+0.26X₁−0.23X₂−0.08X₁²+0.03X₂²+0.22X₁X₂

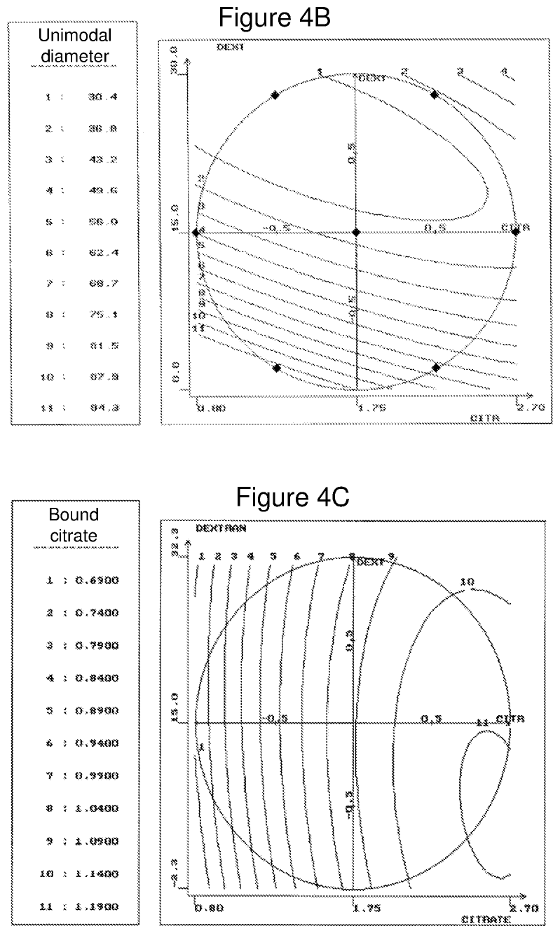

The equation shows that the main effects are related to citrate (increased pH when the concentration increases), to dextran-T1 (decrease when the concentration increases) and to the interaction of both factors. The response curves (FIG. 4A) illustrate these results, without showing any characteristic optimum. At high temperature, citrate acts as a buffer, allowing limitation of the decrease in pH related to the presence of dextran. This phenomenon is not visible in normal conditions for storage.

### Unimodal Diameter

The response of the mean unimodal diameter (25° C.) is modelled by the following polynomial:

Diameter=34.1−8.3X₁−30.5X₂+7.0X₁²+29.2X₂²+20.1X₁X₂

The equation shows that dextran has the major effect. There is a slight interaction between citrate and dextran-T1. The response curves (FIGS. 4B) clearly show an optimal zone for dextran-T1 contents from 20 to 30 mg/g and for citrate contents inferior to 2.6 mg/g.

The response of mean unimodal diameter (55° C.) is modelled by the following polynomial :

Diameter=37.9−13.8X₁−57.8X₂+12.1X₁²+55.2X₂²+30.5X₁X₂

The effects are more marked than at 25° C., but their nature is unchanged.

### Zeta Potential

The response of potential is modelled by the following polynomial:

ζ=−42.5−6.1X₁−3.3X₂+11.7X₁²−0.5X₂²+3.1X₁X₂

The equation shows that citrate has the major effect. The optimum (maximum load) is within a range comprised between 1.4 and 2.3 mg/g, concentrations above 2.3 mg/g (12.12 wt.-% vs iron) have no further effect.

### Bound Citrate

The response of bound citrate is modelled by the following polynomial:

Bound citrate=1.09+0.25X₁−0.01X₂−0.15X₁²−0.04X₂²−0.03X₁X₂

As expected, the equation shows that only citrate has an effect on this parameter. The response curves (FIG. 4C) also show a phenomenon of saturation in citrate for the particle surface. The optimal response is from 0 to 1.1 mg/g, and it is obtained from concentrations in total citrate equal to 1.4 mg/g. Above 2.1-2.2 mg/g the excess total citrate has no more significant effect. These results are consistent with those obtained for the zeta potential.

### Conclusion

The study showed that citrate contributes to particle charge and it is beneficial at a concentration superior to 1.4 mg/g; dextran-T1 acts as a cryoprotector during lyophilization and is most efficient at a concentration superior or equal to 20 mg/g.

### Example 4—Lyophilisation of dUSPIO

Three different compositions were analysed for their thermal properties using standard techniques for monitoring ice matrix changes by examining electrical conduction properties. The composition comprising both dextran-T1 and citrate was found to be more resistant to lyophilisation, beyond what could be expected based on the additive effect of the individual additives. This translates into a slower and more gradual lyophilisation, which reduces particle aggregation.

TABLE 5
thermic properties of compositions as described herein

| | | | | | Dextran + | |
|---|---|---|---|---|---|---|
| Parameter | Dextran | | Citrate | | citrate | |
| Iron content | 20 | mg/mL | 20 | mg/mL | 20 | mg/mL |
| Dextran-T1 | 20 | mg/mL | — | | 20 | mg/mL |
| Citrate | — | | 25 | mM | 25 | mM |
| Freezing temperature | −50° | C. | −35° | C. | −50° | C. |
| Collapse temperature | −13° | C. | −20° | C. | −43° | C. |
| Incipient melting | −4° | C. | −10° | C. | −9° | C. |
| temperature | | | | | | |
| Sublimation | −18° | C. | −25° | C. | −48° | C. |
| temperature | | | | | | |
| Recommended | 200 | mTorr | 200 | mTorr | 20 | mTorr |

| pressure for | | | | | |
|---|---|---|---|---|---|
| lyophilisation | | | | | |
| Recommended | 12 | h | 10 | h | 20 + h |
| duration | | | | | |

of lyophilisation

References

| EP0713602B1 | U.S. Pat. No. | WO 97/25073 | WO 96/09840 |
|---|---|---|---|
| | 5,262,176 | | |

Anzai et al., 1994, AJNR Am J Neuroradiol 15:87-94
Dubois et al. (1956, Anal. Chem., 28:350-356)
Corot et al., doi:10.1016/j.addr.2006.09.013
Laurent et al., doi: 10.1021/cr068445e
Jung, 1995, Magnetic Resonance Imaging, Vol. 13, No. 5, pp. 675-691
Jung and Jacobs, 1995, Magnetic Resonance Imaging, Vol. 13, No. 5, pp. 661-674
Merup & Hansen, 2007, “Superparamagnetic particles”, doi: 10.1002/9780470022184.hmm409
Paul et al., doi: 10.1021/bc034194u Saleh et al., doi:10.1002/nbm.881
Sautour et al., J. App. Microbiol., 2001, 91, 900-906
Sigal et al., doi: 10.1007/s003300101130 Weinstein et al., doi:10.1038/jcbfm.2009.192
Unterweger et al., doi: 10.2147/IJN.S138108

1. Method for producing an adjusted nanoparticle composition, the method comprising the steps of:
i) providing an ultrafiltrated composition comprising ultra-small superparamagnetic iron oxide nanoparticles coated with dextran-T10;
ii) determining the amount of dextran-T10 present in the ultrafiltrated composition of step i);
iii) adding an amount of dextran-T10 to the ultrafiltrated composition of step i) to obtain an adjusted composition comprising at least 140 and at most 160 wt.-% of dextran-T10 relative to the wt.-% of iron; and
iv) optionally filtering the adjusted composition.

2. The method according to claim 1, wherein the ultrafiltrated composition of step i) or the adjusted composition of step iii) comprises from 1.5-2.5 wt.-% iron.

3. The method according to claim 1, wherein step ii) further comprises determining the amount of iron in the ultrafiltrated composition of step i).

4. The method according to claim 1, wherein step iii) further comprises adding a tonicity agent such as a citrate to the ultrafiltrated composition or to the adjusted composition, preferably in an amount of 7-12 wt.-% relative to the wt.-% of iron.

5. The method according to claim 1, wherein the ultrafiltrated composition of step i) further comprises a pharmaceutically acceptable excipient.

6. The method according to claim 1, wherein step iii) further comprises adding an amount of dextran-T1 to the ultrafiltrated composition or to the adjusted composition, so that the adjusted composition comprises at least 140 and at most 160 wt.-% dextran-T1 relative to the wt.-% of iron.

7. The method according to claim 6, wherein the adjusted composition comprises a substantially equal amount by weight of dextran-T10 and of dextran-T1.

8. The method according to claim 1, wherein in step
i) the ultrafiltrated composition is provided by a method comprising the steps of:
i.a) providing a solution of dextran-T10, FeCl₃, and FeCl₂ in water;
i.b) adding a base such as ammonium hydroxide to the solution of step i.a);
i.c) heating the solution of step i.b) to over 60° C. to obtain a nanoparticle composition comprising ultra-small superparamagnetic iron oxide nanoparticles coated with dextran-T10;
i.d) purifying the nanoparticle composition of step i.c) using ultrafiltration to obtain an ultrafiltrated composition.

9. The method according to claim 1, wherein a filtrate of the ultrafiltrated composition of step i) has a conductivity of less than 500 μS/cm, preferably of less than 50 μS/cm, more preferably of less than 25 μS/cm.

10. The method according to claim 1, further comprising the step of lyophilizing the adjusted composition.

11. Composition comprising:
i) ultra-small superparamagnetic iron oxide nanoparticles coated with dextran-T10;
ii) free dextran-T10;
iii) optionally a tonicity agent such as a citrate;
wherein the composition comprises at least 140 and at most 160 wt.-% of dextran-T10 relative to the wt.-% of iron.

12. (canceled)

13. The composition according to claim 11, wherein the concentration of dextran-T10 has a dispersion of at most ±10%.

14. The composition according to claim 11, wherein the unimodal mean diameter of the nanoparticles is stable for at least 6 months.

15. (canceled)

16. A method of performing an MRI, comprising administering a contrast agent to a subject, wherein the contrast agent is the composition according to claim 11.
